(19) United States
(12) Patent Application Publication
(10) Pub. No.: US 2025/0208080 A1
(43) Pub. Date: Jun. 26, 2025

(54) HYDROGEN DETECTION ELEMENT AND METHOD FOR MANUFACTURING THE SAME

(71) Applicant: Nuvoton Technology Corporation Japan, Kyoto (JP)

(72) Inventors: Tomohiro MINAGAWA, Shiga (JP); Satoru ITO, Hyogo (JP); Ken KAWAI, Osaka (JP); Kazunari HOMMA, Gifu (JP); Koji KATAYAMA, Nara (JP)

(21) Appl. No.: 19/058,748

(22) Filed: Feb. 20, 2025

Related U.S. Application Data
(63) Continuation of application No. PCT/JP2023/031689, filed on Aug. 31, 2023.
(60) Provisional application No. 63/404,049, filed on Sep. 6, 2022.

(51) Int. Cl. G01N 27/12; G01N 33/00

(52) U.S. Cl. CPC G01N 27/125; G01N 33/005

(57) ABSTRACT

A hydrogen detection element includes: a first electrode that is planar; a second electrode that is planar, is disposed opposite to the first electrode, includes a principal surface covered by an insulating film (a protective film and a second insulating film), and includes a plurality of exposed portions that serve as a plurality of hydrogen gas inlets and are each provided by opening part of the insulating film on the principal surface; a metal oxide layer that is disposed between the first electrode and the second electrode; and a first terminal and a second terminal that are electrically connected to the second electrode at positions between which the plurality of exposed portions are arranged in plan view of the second electrode. When hydrogen gas is introduced to the plurality of exposed portions, resistance between the first terminal and the second terminal changes.

### CROSS REFERENCE TO RELATED APPLICATION

This is a continuation application of PCT Patent Application No. PCT/JP2023/031689 filed on Aug. 31, 2023, designating the United States of America, which is based on and claims priority of U.S. Provisional Patent Application No. 63/404,049 filed on Sep. 6, 2022. The entire disclosures of the above-identified applications, including the specifications, drawings and claims are incorporated herein by reference in their entirety.

### FIELD

The present disclosure relates to a hydrogen detection element and a manufacturing method thereof, and particularly relates to a hydrogen detection element that has a structure in which a metal oxide layer is disposed between two electrodes.

### BACKGROUND

A hydrogen detection element that has a structure in which a metal oxide layer is disposed between two electrodes has been conventionally proposed (see Patent Literature (PTL) 1, for example).

The hydrogen detection element according to PTL 1 has a structure in which a first electrode, a metal oxide layer, a second electrode, and an insulating film are stacked in this order from the bottom. An exposed portion serving as a hydrogen gas inlet to the second electrode is formed by opening part of the insulating film. The concentration of hydrogen gas is detected by utilizing the fact that the resistance value of the hydrogen detection element changes according to the concentration of the hydrogen gas introduced to the exposed portion.

### CITATION LIST

### Patent Literature

PTL 1: WO 2018/123674

### SUMMARY

### Technical Problem

However, individual variation in reaction characteristic with respect to hydrogen (i.e., amount of change in resistance value) occurs among hydrogen detection elements according to PTL 1. It should be noted that “individual variation” means variation in characteristic among individual hydrogen detection elements manufactured, and is also simply referred to as “variation” hereinafter.

The present disclosure provides: a hydrogen detection element that has a characteristic structure for suppressing variation in reaction characteristic; and a manufacturing method of the same.

### Solution to Problem

A hydrogen detection element according to an aspect of the present disclosure includes: a first electrode that is planar; a second electrode that is planar, is disposed opposite to the first electrode, includes a principal surface covered by an insulating film, and includes a plurality of exposed portions that each serve as a hydrogen gas inlet and are each provided by opening part of the insulating film on the principal surface; a metal oxide layer that is disposed between the first electrode and the second electrode; and a first terminal and a second terminal that are electrically connected to the second electrode at positions between which the plurality of exposed portions are arranged in plan view of the second electrode. When hydrogen gas is introduced to the plurality of exposed portions, resistance between the first terminal and the second terminal changes.

A method for manufacturing a hydrogen detection element according to an aspect of the present disclosure includes: forming a first electrode that is planar; forming a metal oxide layer on the first electrode; forming a second electrode on the metal oxide layer; forming a first terminal and a second terminal that are electrically connected to the second electrode; forming an insulating film that covers the second electrode; and forming, on a principal surface of the second electrode, a plurality of exposed portions that serve as a plurality of hydrogen gas inlets, by removing a plurality of portions of the insulating film between the first terminal and the second terminal in plan view of the second electrode. When hydrogen gas is introduced to the plurality of exposed portions, resistance between the first terminal and the second terminal changes.

### Advantageous Effects

The present disclosure provides: a hydrogen detection element that has a characteristic structure for suppressing variation in reaction characteristic; and a manufacturing method of the same.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 illustrates the structure of a hydrogen detection element according to an embodiment.

FIG. 2A illustrates variations of the number, shape, and arrangement position of a plurality of exposed portions included in a single hydrogen detection element according to the embodiment.

FIG. 2B illustrates other variations of the number, shape, and arrangement position of a plurality of exposed portions included in a single hydrogen detection element according to the embodiment.

FIG. 2C illustrates other variations of the number, shape, and arrangement position of a plurality of exposed portions included in a single hydrogen detection element according to the embodiment.

FIG. 3A is a cross-sectional view illustrating a method for manufacturing a hydrogen detection element according to the embodiment.

FIG. 3B is a cross-sectional view illustrating the method for manufacturing the hydrogen detection element according to the embodiment (continuation of FIG. 3A).

FIG. 3C is a cross-sectional view illustrating the method for manufacturing the hydrogen detection element according to the embodiment (continuation of FIG. 3B).

FIG. 3D is a cross-sectional view illustrating the method for manufacturing the hydrogen detection element according to the embodiment (continuation of FIG. 3C).

FIG. 4 illustrates the result of an experiment regarding variation in reaction characteristic among conventional hydrogen detection elements and variation in reaction characteristic among hydrogen detection elements according to the embodiment.

FIG. 5A illustrates, for each wafer, a distribution of variation in sensor resistance among hydrogen detection elements each of which includes a main body having the size of a 5 μm □ (a 5 μm by 5 μm square) and a single exposed portion having the size of a 3 μm □.

FIG. 5B illustrates, for each wafer, a distribution of variation in sensor resistance among hydrogen detection elements each of which includes a main body having the size of a 3 μm □ and a single exposed portion having the size of a 1.8 μm □.

FIG. 5C illustrates, for each wafer, a distribution of variation in sensor resistance among hydrogen detection elements each of which includes a main body having the size of a 2 μm □ and a single exposed portion having the size of a 1.2 μm □.

FIG. 5D illustrates, for each wafer, a distribution of variation in sensor resistance among hydrogen detection elements each of which includes a main body having the size of a 1.5 μm □ and a single exposed portion having the size of a 0.9 μm □.

FIG. 5E illustrates, for each wafer, a distribution of variation in sensor resistance among hydrogen detection elements each of which includes a main body having the size of a 5 μm □ and four exposed portions each having the size of a 1.5 μm □.

FIG. 5F illustrates, for each wafer, a distribution of variation in sensor resistance among hydrogen detection elements each of which includes a main body having the size of a 5 μm □ and nine exposed portions each having the size of a 1 μm □.

FIG. 6 illustrates the result of an experiment regarding dependency between dimension of exposed portion in second direction and variation in sensor resistance among hydrogen detection elements according to the embodiment.

FIG. 7 illustrates arrangement examples of a plurality of exposed portions included in a single hydrogen detection element according to the embodiment, reflecting the knowledge obtained from the result of the experiment shown in FIG. 6.

FIG. 8 is a diagram for describing the result of an experiment regarding dependency between dimension of opening of exposed portion and variation in resistance among hydrogen detection elements each of which includes exposed portion in square pattern.

FIG. 9 is a diagram for describing the result of an experiment regarding a total opening area of exposed portions included in a single hydrogen detection element according to the embodiment.

### DESCRIPTION OF EMBODIMENT

### Knowledge Obtained by the Inventors

After investigating the cause of variation in reaction characteristic among hydrogen detection elements disclosed in PTL 1, the inventors have found that variation in reaction characteristic among hydrogen detection elements are caused depending on the quality of an exposed portion that is formed by opening part of an insulating film and serves as a hydrogen gas inlet to a second electrode. In other words, the inventors have found that when part of an insulating film is opened by dry etching, a Pt layer that is included in a second electrode and remains on an exposed portion of the second electrode may vary among hydrogen detection elements, and variation in reaction characteristic among the hydrogen detection elements is caused by the variation in remaining Pt layer among the hydrogen detection elements.

Then, the inventors manufactured a hydrogen detection element while making an opening size of an insulating film smaller than that of a conventional hydrogen detection element. Thus, it was confirmed that variation in reaction characteristic was suppressed. However, when the area of an opening is reduced, an amount of reaction to hydrogen is reduced and a sensor characteristic (i.e., sensitivity to hydrogen) of a hydrogen detection element is deteriorated.

Then, the inventors manufactured a hydrogen detection element while making an opening size of an insulating film smaller than that of a conventional hydrogen detection element and providing, in contrast to the conventional hydrogen detection element including a single opening, a plurality of openings to the hydrogen detection element so as to ensure a total area that reacts to hydrogen. As a result, the hydrogen detection element having a characteristic structure for suppressing variation in reaction characteristic was manufactured while maintaining a desired sensor characteristic (i.e., sensitivity to hydrogen).

### Embodiment

Hereinafter, a hydrogen detection element according to the embodiment and a manufacturing method of the same are described with reference to the Drawings. It should be noted that the embodiment described below shows a specific example of the present disclosure. The numerical values, shapes, materials, constituent elements, the arrangement and connection of the constituent elements, steps, the order of the steps, etc., in the following embodiment are mere examples, and therefore do not intend to limit the present disclosure. Moreover, each drawing is not necessarily an exact depiction. In each drawing, elements that are substantially the same share the same reference sign, and overlapping description thereof may be omitted or simplified.

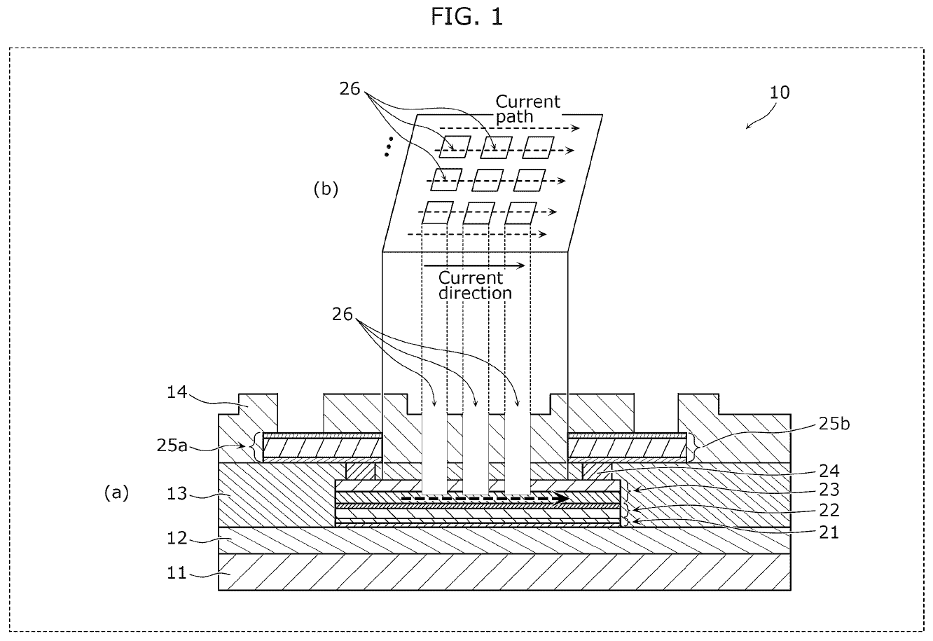

FIG. 1 illustrates the structure of hydrogen detection element 10 according to the embodiment. More specifically, (a) in FIG. 1 is a cross-sectional view illustrating the layered structure of hydrogen detection element 10, and (b) in FIG. 1 is a top view of an area (i.e., exposed portions 26) between first terminal 25a and second terminal 25b of hydrogen detection element 10. It should be noted that in (a) and (b) in FIG. 1, a dashed arrow indicates a direction in which a current flows.

In (a) in FIG. 1, hydrogen detection element 10 has the structure in which semiconductor substrate 11, first insulating film 12, first electrode 21, metal oxide layer 22, second electrode 23, inter-wiring plug 24, first terminal 25a as well as second terminal 25b, and protective film 14 are stacked in this order from the bottom. Second insulating film 13 covers both sides of each of first electrode 21, metal oxide layer 22, second electrode 23, and inter-wiring plug 24, and an upper surface of second electrode 23.

As shown in (b) in FIG. 1, in top view of hydrogen detection element 10, at a plurality of positions (here, nine positions) in an area between first terminal 25a and second terminal 25b, protective film 14 that is the upper most layer, second insulating film 13 that is disposed thereunder, and part (upper layer portion) of second electrode 23 that is disposed thereunder are removed to form a plurality of openings that are a plurality of exposed portions 26 each serving as a hydrogen gas inlet to a principal surface (i.e., upper surface) of second electrode 23. Moreover, part of protective film 14 is removed at part of an upper surface of first terminal 25a and part of an upper surface of second terminal 25b to form a hole for electrically contacting first terminal 25a and a hole for electrically contacting second terminal 25b.

Hydrogen detection element 10 having such a structure is a variable resistance element in which resistance between first terminal 25a and second terminal 25b changes when hydrogen gas is introduced to the plurality of exposed portions 26. In other words, a resistance value between first terminal 25a and second terminal 25b is obtained by applying voltage between first terminal 25a and second terminal 25b of hydrogen detection element 10 to cause a current to flow in the direction illustrated in FIG. 1 in a hydrogen atmosphere in which the plurality of exposed portions 26 are exposed to hydrogen gas. The resistance value obtained corresponds to the concentration of the hydrogen gas.

It should be noted that the minimum constituent elements of hydrogen detection element 10 among the elements shown in FIG. 1 are (1) first electrode 21 that is planar, (2) second electrode 23 that is planar, is disposed opposite to first electrode 21, includes the principal surface covered by the insulating film (protective film 14 and second insulating film 13), and includes the plurality of exposed portions 26 that serve as a plurality of hydrogen gas inlets and are formed by opening part of the insulating film on the principal surface, (3) metal oxide layer 22 that is disposed between first electrode 21 and second electrode 23, and (4) first terminal 25a and second terminal 25b that are electrically connected to second electrode 23 at positions between which the plurality of exposed portions 26 are arranged in plan view of second electrode 23.

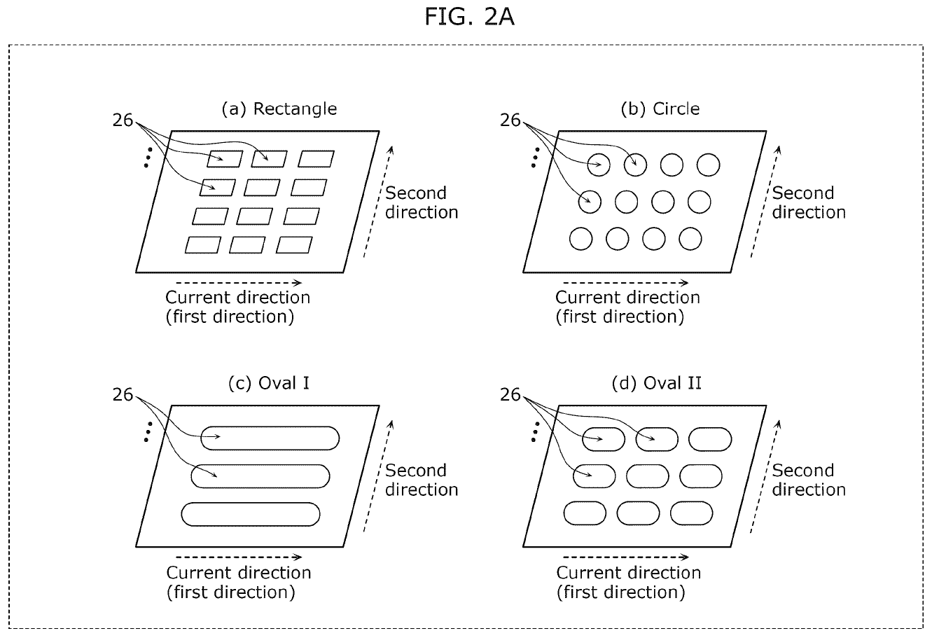

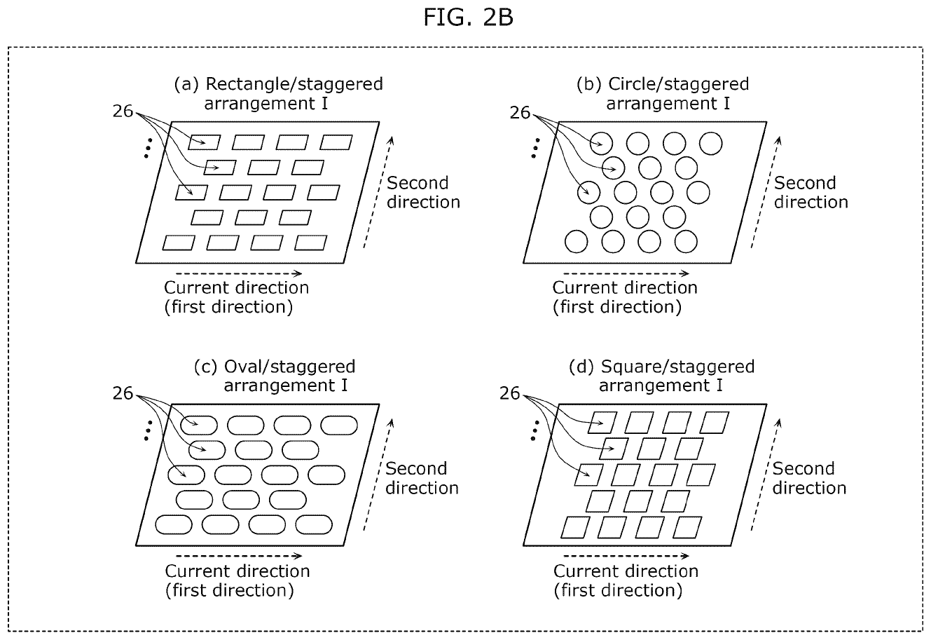

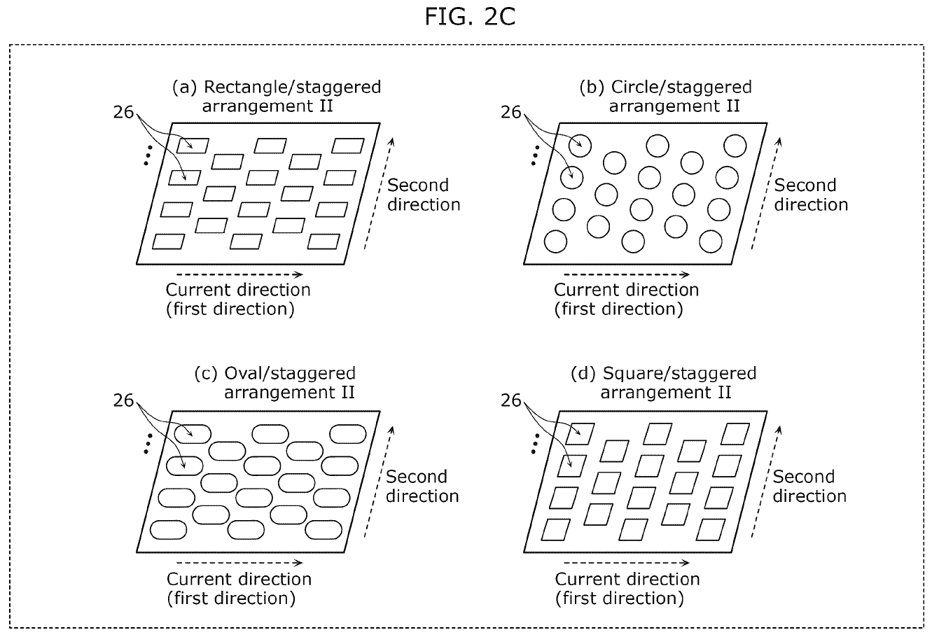

Moreover, the number, shape, and arrangement position of the plurality of exposed portions 26 are not limited to those shown in (b) in FIG. 1. FIG. 2A to FIG. 2C illustrate variations of the number, shape, and arrangement position of the plurality of exposed portions 26 included in a single hydrogen detection element 10 according to the embodiment. It should be noted that in each of FIG. 2A to FIG. 2C, a current direction connecting first terminal 25a and second terminal 25b is referred to as a first direction, and a direction that is perpendicular to the first direction and parallel to the principal surface of second electrode 23 is referred to as a second direction.

In FIG. 2A, the plurality of exposed portions 26 are arranged in straight lines both in the first direction and the second direction, and arranged in a grid as a whole. More specifically, in (a) in FIG. 2A, total twelve exposed portions 26 each of which is in a rectangular shape elongated in the first direction are arranged in columns and rows, and each of the rows includes three exposed portions 26 aligned in the first direction and each of the columns includes four exposed portions 26 aligned in the second direction. In (b) in FIG. 2A, total twelve exposed portions 26 each of which is in a circular shape are arranged in columns and rows, and each of the rows includes four exposed portions 26 aligned in the first direction and each of the columns includes three exposed portions 26 aligned in the second direction. In (c) in FIG. 2A, total three exposed portions 26 each of which is in an oval shape elongated in the first direction (“Oval I”) are aligned in the second direction. In (d) in FIG. 2A, total nine exposed portions 26 each of which is in an oval shape elongated in the first direction (“Oval II”) are arranged in columns and rows, and each of the rows includes three exposed portions 26 aligned in the first direction and each of the columns includes three exposed portions 26 aligned in the second direction.

In FIG. 2B, the plurality of exposed portions 26 are arranged in straight lines both in the first direction and the second direction, and arranged, as a whole, in a staggered arrangement in which exposed portions 26 are staggered relative to the adjacent rows extending in the first direction (“staggered arrangement I”). More specifically, in (a) in FIG. 2B, total eighteen exposed portions 26 each of which is in a rectangular shape elongated in the first direction are arranged in a pattern in which a row including four exposed portions 26 aligned in the first direction and a row including three exposed portions 26 aligned in the first direction are alternately arranged in the second direction. In (b) in FIG. 2B, total eighteen exposed portions 26 each of which is in a circular shape are arranged in a pattern in which a row including four exposed portions 26 aligned in the first direction and a row including three exposed portions 26 aligned in the first direction are alternately arranged in the second direction. In (c) in FIG. 2B, total eighteen exposed portions 26 each of which is in an oval shape elongated in the first direction are arranged in a pattern in which a row including four exposed portions 26 aligned in the first direction and a row including three exposed portions 26 aligned in the first direction are alternately arranged in the second direction. In (d) in FIG. 2B, total eighteen exposed portions 26 each of which is in a square shape are arranged in a pattern in which a row including four exposed portions 26 aligned in the first direction and a row including three exposed portions 26 aligned in the first direction are alternately arranged in the second direction.

In FIG. 2C, the plurality of exposed portions 26 are arranged in straight lines both in the first direction and the second direction, and arranged, as a whole, in a staggered arrangement in which exposed portions 26 are staggered relative to the adjacent columns extending in the second direction (“staggered arrangement II”). More specifically, in (a) in FIG. 2C, total eighteen exposed portions 26 each of which is in a rectangular shape elongated in the first direction are arranged in a pattern in which a column including four exposed portions 26 aligned in the second direction and a column including three exposed portions 26 aligned in the second direction are alternately arranged in the first direction. In (b) in FIG. 2C, total eighteen exposed portions 26 each of which is in a circular shape are arranged in a pattern in which a column including four exposed portions 26 aligned in the second direction and a column including three exposed portions 26 aligned in the second direction are alternately arranged in the first direction. In (c) in FIG. 2C, total eighteen exposed portions 26 each of which is in an oval shape elongated in the first direction are arranged in a pattern in which a column including four exposed portions 26 aligned in the second direction and a column including three exposed portions 26 aligned in the second direction are alternately arranged in the first direction. In (d) in FIG. 2C, total eighteen exposed portions 26 each of which is in a square shape are arranged in a pattern in which a column including four exposed portions 26 aligned in the second direction and a column including three exposed portions 26 aligned in the second direction are alternately arranged in the first direction.

It should be noted that the shape of the plurality of exposed portions 26 included in a single hydrogen detection element 10 according to the embodiment is not limited to a rectangular shape, a circular shape, and an oval shape as shown in FIG. 2A to FIG. 2C, and may be a rhombus shape or another polygonal shape.

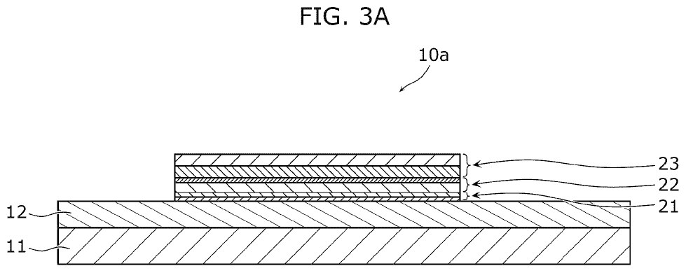

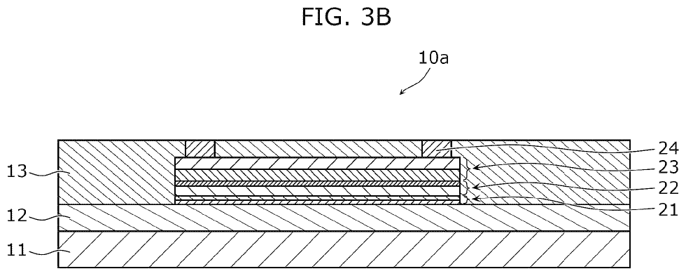

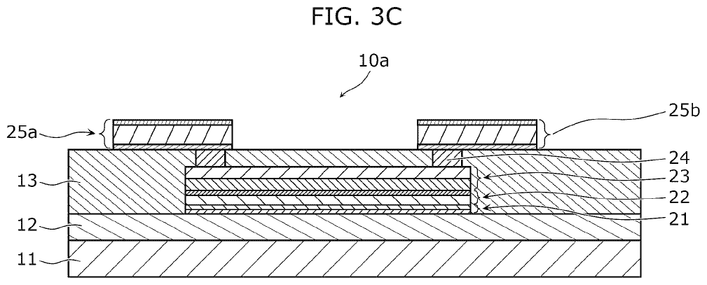

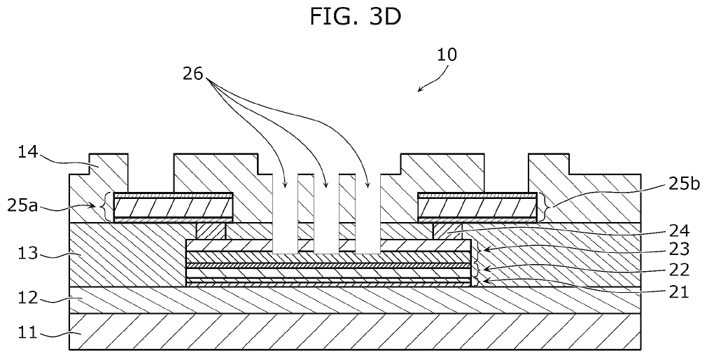

FIG. 3A to FIG. 3D are cross-sectional views illustrating a method for manufacturing hydrogen detection element 10 according to the embodiment.

First, as illustrated in FIG. 3A, for example, a layer of first electrode 21 that includes a metal compound such as TiN or TaN; metal oxide layer 22 that includes, for example, Ta₂O₅ or a layered structure of TaOₓ and Ta₂O₅; and a layer of second electrode 23 that includes, for example, a layered structure of Pt and TIN are formed, by a sputtering method or the like, on first insulating film 12, such as P-TEOS, that is formed on semiconductor substrate 11 by chemical vapor deposition (CVD) method or the like. Then, the layer of first electrode 21, metal oxide layer 22, and the layer of second electrode 23 are processed into a desired pattern by a photolithography method, a dry etching method, or the like to form main body 10a of a hydrogen detection element.

Next, as illustrated in FIG. 3B, main body 10a of the hydrogen detection element formed is covered by second insulating film 13 such as P-TEOS by the CVD method or the like, hole-like openings are formed by a lithography method, a dry etching method, or the like, each of the hole-like openings is filled with a layered structure of TiN and W or the like by the CVD method or the like, and an unnecessary portion is removed by a chemical mechanical polishing (CMP) method, etch-back method, or the like, to form inter-wiring plugs 24 that cause second electrode 23 of the hydrogen detection element to be electrically connected to first terminal 25a and second terminal 25b that are formed later.

Next, as illustrated in FIG. 3C, a metal film such as a layered film of AlCu, TiN, and Ti is formed on inter-wiring plugs 24 and second insulating film 13 by the sputtering method or the like, and the metal film is processed into a desired pattern by a photolithography method, a dry-etching method, or the like to form first terminal 25a and second terminal 25b. Thus, second electrode 23 of the hydrogen detection element is electrically connected to first terminal 25a and second terminal 25b through inter-wiring plugs 24.

Then, as illustrated in FIG. 3D, protective film 14 such as P-SiON is formed by the CVD method or the like, and protective film 14 is processed into a desired pattern by a photolithography method, a dry-etching method, or the like to form protective film 14 that includes openings at part of first terminal 25a and part of second terminal 25b. Finally, part of each of second electrode 23, second insulating film 13, and protective film 14 on main body 10a of the hydrogen detection element is removed to form a plurality of exposed portions 26 that are openings through each of which the Pt layer that serves as a catalyst layer for detecting hydrogen and is included in second electrode 23 is exposed. Thus, hydrogen detection element 10 is completed.

Next, a reaction characteristic, with respect to hydrogen, of hydrogen detection device 10 according to the embodiment manufactured as above is described.

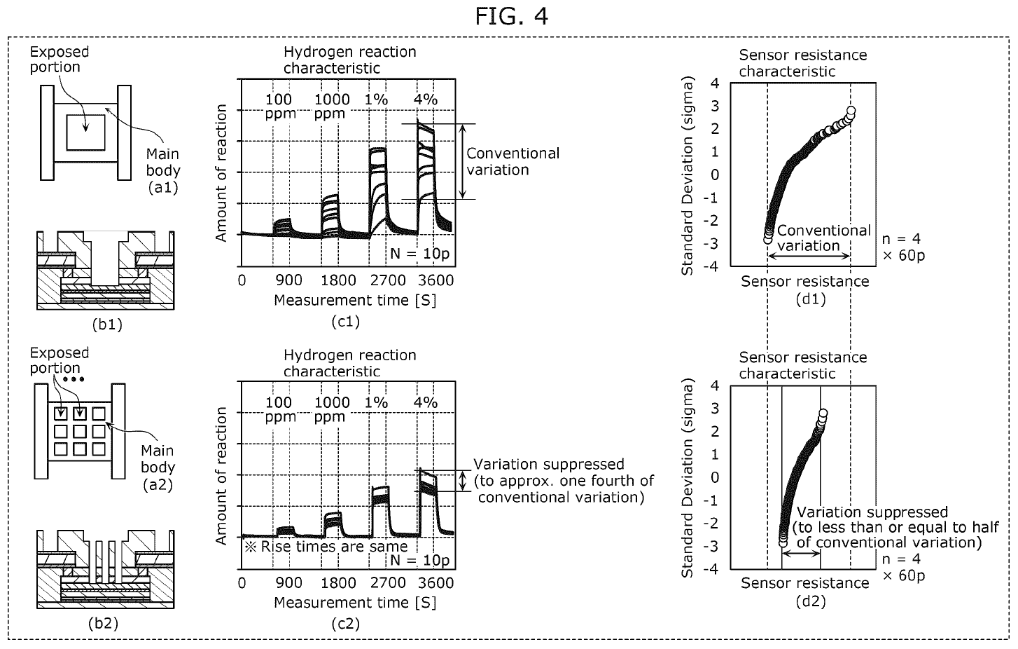

FIG. 4 illustrates the result of an experiment regarding variation in reaction characteristic among conventional hydrogen detection elements and variation in reaction characteristic among hydrogen detection elements according to the embodiment. Here, FIG. 4 shows variation in reaction characteristic measured regarding the conventional hydrogen detection elements and the hydrogen detection elements according to the embodiment, and the conventional hydrogen detection elements and the hydrogen detection elements according to the embodiment were manufactured in the same manufacturing process except for formation of an exposed portion.

More specifically, (a1) in FIG. 4 shows a top view of a conventional hydrogen detection element used in the experiment, (b1) in FIG. 4 shows a cross-sectional view of the conventional hydrogen detection element used in the experiment, (c1) in FIG. 4 shows an experimental result indicating a hydrogen reaction characteristic (temporal change in an amount of reaction) of the conventional hydrogen detection elements (the horizontal axis represents measurement time, the vertical axis represents amount of reaction, and the number of measurement samples is 10), and (d1) in FIG. 4 shows variation in resistance characteristic among the hydrogen detection elements that has a correlation with the hydrogen reaction characteristic (amount of reaction) obtained in (c1) in FIG. 4 (the horizontal axis represents sensor resistance, the vertical axis represents standard deviation, and the number of measurement samples is 240).

(a2) in FIG. 4 shows a top view of a hydrogen detection element according to the embodiment used in the experiment, (b2) in FIG. 4 shows a cross-sectional view of the hydrogen detection element according to the embodiment used in the experiment, (c2) in FIG. 4 shows an experimental result indicating a hydrogen reaction characteristic (temporal change in an amount of reaction) of the hydrogen detection elements according to the embodiment (the horizontal axis represents measurement time, the vertical axis represents amount of reaction, and the number of measurement samples is 10), and (d2) in FIG. 4 shows variation in resistance characteristic among the hydrogen detection elements that has a correlation with the hydrogen reaction characteristic (amount of reaction) obtained in (c2) in FIG. 4 (the horizontal axis represents sensor resistance, the vertical axis represents standard deviation, and the number of measurement samples is 240).

As illustrated in (a1) and (b1) in FIG. 4, the conventional hydrogen detection element used in the experiment includes a single exposed portion having a large size (3 μm □) formed by opening the upper surface of a main body. In contrast, as illustrated in (a2) and (b2) in FIG. 4, the hydrogen detection element according to the embodiment used in the experiment includes, on the upper surface of a main body, nine exposed portions each having a smaller size (1 μm □) than that of the exposed portion of the conventional hydrogen detection element. It should be noted that “□” means a square. The total opening area of the exposed portion of the conventional hydrogen detection element and the total opening area of the exposed portions of the hydrogen detection element according to the embodiment are the same 9 μm². It should be noted that the main body corresponds to an area of the second electrode between a first terminal and a second terminal in top view of the hydrogen detection element.

Each of (c1) and (c2) in FIG. 4 shows temporal change in sensor resistance (amount of reaction) of each of hydrogen detection elements when an exposed portion of the hydrogen detection element was exposed to hydrogen at a concentration of 100 ppm, 1000 ppm, 1%, and 4% in this order in a pulsing manner in the experiment regarding hydrogen reaction characteristic. As can be seen from comparison of (c1) and (c2) in FIG. 4, the variation in amount of reaction among the hydrogen detection elements according to the embodiment was suppressed to approx. one fourth of the variation in amount of reaction among the conventional hydrogen detection elements.

As can be seen from comparison of (d1) and (d2) in FIG. 4, the variation in sensor resistance among the hydrogen detection elements according to the embodiment was suppressed to approx. one half of the variation in sensor resistance among the conventional hydrogen detection elements.

In view of this, it can be seen that variation in reaction characteristic is suppressed by making the size of each exposed portion small and providing a plurality of exposed portions to a hydrogen detection element without changing the total opening area.

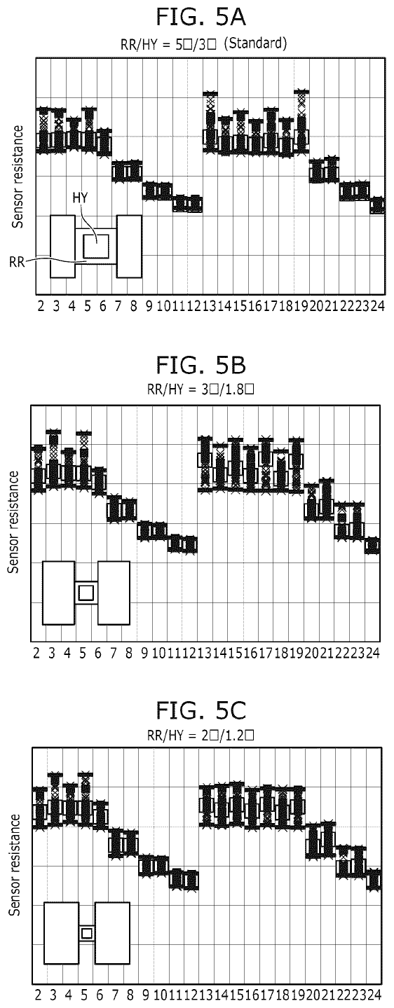

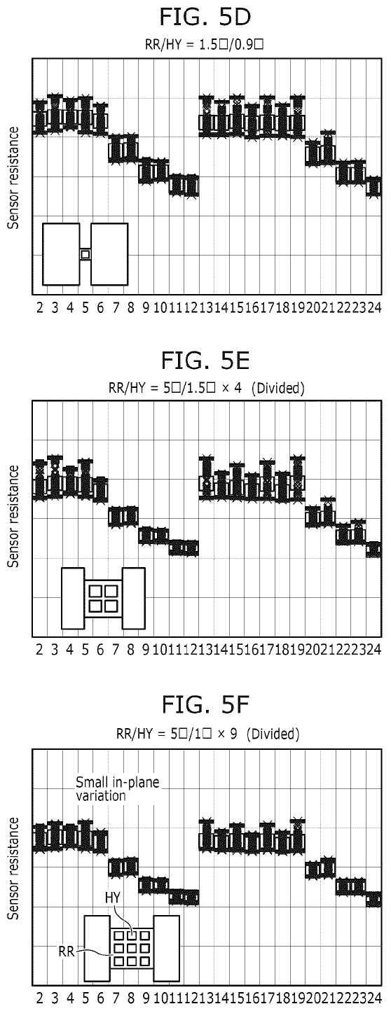

Each of FIG. 5A to FIG. 5F is a diagram illustrating the result of an experiment regarding variation in sensor resistance among hydrogen detection elements each of which includes a main body having a predetermined size and a predetermined number of exposed portions each having a predetermined size. The horizontal axis represents slice No. for identifying a wafer from which corresponding hydrogen detection elements were manufactured, and the vertical axis represents sensor resistance value measured for each of hydrogen detection elements obtained from a corresponding wafer. Here, since 60 hydrogen detection elements were sampled from each wafer, the measured values (× marks), median value (rectangle box), maximum value (horizontal bar), and minimum value (horizontal bar) of sensor resistance of the 60 hydrogen detection elements are shown for each slice No. on the horizontal axis. At each of the upper side and lower-left side of the graph, the size of a main body (“RR”) and the size and number of exposed portions (“HY”) of a hydrogen detection element are shown. It should be noted that since the wafers were manufactured while intentionally making the Pt layer thickness different between a group of slice Nos. 2 to 6 and 13 to 19, a group of slice Nos. 7, 8, 20, and 21, a group of slice Nos. 9, 10, 22, and 23, and a group of slice Nos. 11, 12, and 24, the median value of the sensor resistance is different between the groups.

More specifically, FIG. 5A illustrates a distribution of variation in sensor resistance per wafer (i.e., for each 60 hydrogen detection elements), and each hydrogen detection element includes a main body (“RR”) having the size of a 5 μm □ and a single exposed portion (“HY”) having the size of a 3 μm □ as illustrated in a pattern diagram in the lower-left part of FIG. 5A. It should be noted that the structure of the hydrogen detection element illustrated in the figure is the same as that of the conventional hydrogen detection element illustrated in (a1) and (b1) in FIG. 4.

FIG. 5B illustrates a distribution of variation in sensor resistance per wafer (i.e., for each 60 hydrogen detection elements), and each hydrogen detection element includes a main body (“RR”) having the size of a 3 μm □ and a single exposed portion (“HY”) having the size of a 1.8 μm □ as illustrated in a pattern diagram in the lower-left part of FIG. 5B.

FIG. 5C illustrates a distribution of variation in sensor resistance per wafer (i.e., for each 60 hydrogen detection elements), and each hydrogen detection element includes a main body (“RR”) having the size of a 2 μm □ and a single exposed portion (“HY”) having the size of a 1.2 μm □ as illustrated in a pattern diagram in the lower-left part of FIG. 5C.

FIG. 5D illustrates a distribution of variation in sensor resistance per wafer (i.e., for each 60 hydrogen detection elements), and each hydrogen detection element includes a main body (“RR”) having the size of a 1.5 μm □ and a single exposed portion (“HY”) having the size of a 0.9 μm □ as illustrated in a pattern diagram in the lower-left part of FIG. 5D.

FIG. 5E illustrates a distribution of variation in sensor resistance per wafer (i.e., for each 60 hydrogen detection elements), and each hydrogen detection element includes a main body (“RR”) having the size of a 5 μm □ and four exposed portions (“HY”) each having the size of a 1.5 μm □ as illustrated in a pattern diagram in the lower-left part of FIG. 5E.

FIG. 5F illustrates a distribution of variation in sensor resistance per wafer (i.e., for each 60 hydrogen detection elements), and each hydrogen detection element includes a main body (“RR”) having the size of a 5 μm □ and nine exposed portions (“HY”) each having the size of a 1 μm □ as illustrated in a pattern diagram in the lower-left part of FIG. 5F. It should be noted that the structure of the hydrogen detection element illustrated in the figure is the same as that of the hydrogen detection element according to the embodiment illustrated in (a2) and (b2) in FIG. 4.

When focusing on the size of a single exposed portion (“HY”) and comparing, in descending order of size, FIG. 5A illustrating the case in which the size of an exposed portion is a 3 μm □, FIG. 5B illustrating the case in which the size of an exposed portion is a 1.8 μm □, FIG. 5E illustrating the case in which the size of an exposed portion is a 1.5 μm □, FIG. 5C illustrating the case in which the size of an exposed portion is a 1.2 μm □, FIG. 5F illustrating the case in which the size of an exposed portion is a 1 μm □, and FIG. 5D illustrating the case in which the size of an exposed portion is a 0.9 μm □, it can be seen that there is a tendency that the smaller a single exposed portion (“HY”) is, the smaller the variation in sensor resistance is.

Moreover, when focusing on difference in number of exposed portions (“HY”) between FIG. 5A illustrating the case in which the number of exposed portions (“HY”) is 1 and FIG. 5F illustrating the case in which the number of exposed portions (“HY”) is 9, although the size of a main body (“RR”) is the same 5 μm □ and the total opening area is the same 9 μm² between FIG. 5A and FIG. 5F, it can be seen, from comparison of FIG. 5A and FIG. 5F, that variation in reaction characteristic can be suppressed by making the size of each exposed portion small and providing a plurality of exposed portions to a single hydrogen detection element without changing the total opening area.

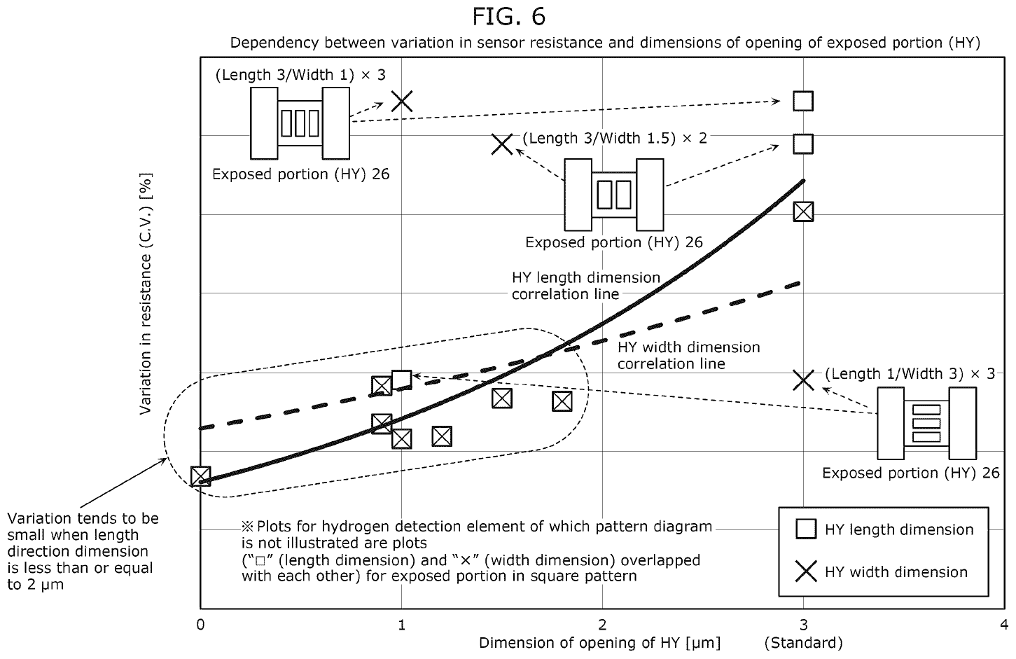

FIG. 6 illustrates the result of an experiment regarding dependency between dimension of exposed portion 26 in second direction and variation in sensor resistance among hydrogen detection elements according to the embodiment. The horizontal axis represents dimension (μm) of opening of exposed portion (“HY”) 26, and the vertical axis represents variation (Coefficient of Variation (C.V.): percentage (%) of standard deviation with respect to mean) in resistance measured for 60 hydrogen detection elements that include exposed portions 26 and have been manufactured from each wafer. In the graph, “□” represents the length dimension of exposed portion (“HY”) 26, and “X” represents the width dimension of exposed portion (“HY”) 26. Here, “width” of “width dimension” means the first direction (i.e., the current direction connecting first terminal 25a and second terminal 25b), and “length” of “length dimension” means the second direction (i.e., the direction that is perpendicular to the first direction and parallel to the principal surface of second electrode 23).

In FIG. 6, variation in resistance is plotted for each pattern of hydrogen detection elements including four types of exposed portions 26. Exposed portion 26 of the first type is exposed portion 26 having dimensions of length 3: width 1, and, as illustrated in a pattern diagram in the upper left part of the graph, hydrogen detection elements each including three exposed portions 26 of the first type were used. Exposed portion 26 of the second type is exposed portion 26 having dimensions of length 3: width 1.5, and, as illustrated in a pattern diagram in the upper middle part of the graph, hydrogen detection elements each including two exposed portions 26 of the second type were used. Exposed portion 26 of the third type is exposed portion 26 having dimensions of length 1: width 3, and, as illustrated in a pattern diagram in the lower right part of the graph, hydrogen detection elements each including three exposed portions 26 of the third type were used. Exposed portion 26 of the fourth type is exposed portion 26 in a square pattern, that is, “□” representing the length dimension of exposed portion (“HY”) 26 of the fourth type and “X” representing the width dimension of exposed portion (“HY”) 26 of the fourth type are overlapped with each other in the graph. Eight patterns of hydrogen detection elements each including one or more exposed portions 26 of the fourth type were used, and a pattern diagram is not illustrated for the fourth type.

Moreover, an approximate curve that represents a correlation between length dimension of exposed portion (“HY”) 26 and variation in resistance (“HY length dimension correlation line”, the solid curve line) and an approximate curve that represents a correlation between width dimension of exposed portion (“HY”) 26 and variation in resistance (“HY width dimension correlation line”, the dashed curve line) are shown in FIG. 6.

In FIG. 6, since the gradient of the “HY length dimension correlation line” is steeper than that of the “HY width dimension correlation line”, it can be seen that variation in resistance among hydrogen detection elements is largely dependent on length direction dimension of exposed portion 26. Moreover, in FIG. 6, the plots for exposed portions 26 in a rectangular pattern (the plots for three patterns of hydrogen detection elements of which pattern diagrams are illustrated in the graph) are significantly away from the “HY width dimension correlation line”. Accordingly, it can be seen that variation in resistance can be suppressed by making the dimension of exposed portion 26 in the length direction (i.e., the second direction orthogonal to the current direction) small. As shown by the “HY length dimension correlation line” and the dashed frame in the figure, the length direction dimension (i.e., the dimension in the second direction) of exposed portion 26 is preferably not greater than 2 μm for suppressing variation in resistance to satisfy the specification realistically required for a hydrogen detection element.

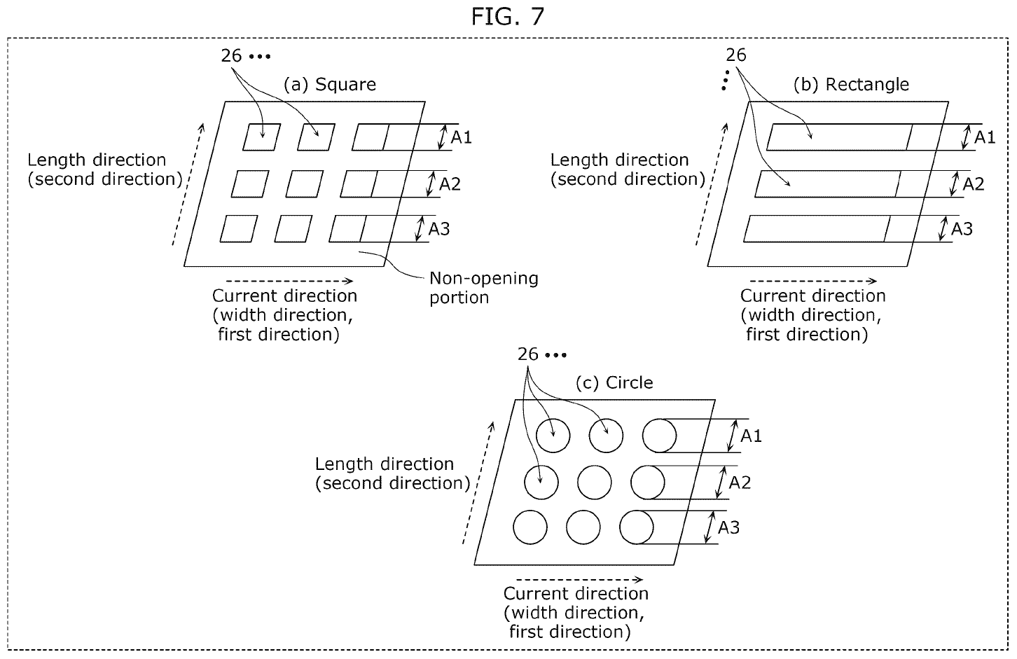

FIG. 7 illustrates arrangement examples of a plurality of exposed portions 26 included in a single hydrogen detection element according to the embodiment, reflecting the knowledge obtained from the result of the experiment shown in FIG. 6 (i.e., the length direction dimension of exposed portion 26 is preferably not greater than 2 μm).

In (a) in FIG. 7, total nine exposed portions 26 each of which is in a square shape are arranged in columns and rows, and each of the rows includes three exposed portions 26 aligned in the current direction (width direction, first direction) and each of the columns includes three exposed portions 26 aligned in the length direction (second direction). In (b) in FIG. 7, total three exposed portions 26 each of which is in a rectangular shape elongated in the current direction (width direction, first direction) are aligned in the length direction (second direction). In (c) in FIG. 7, total nine exposed portions 26 each of which is in a circular shape are arranged in columns and rows, and each of the rows includes three exposed portions 26 aligned in the current direction (width direction, first direction) and each of the columns includes three exposed portions 26 aligned in the length direction (second direction). In each of (a) to (c) in FIG. 7, each of dimensions A1, A2, and A3 that are each the dimension of exposed portion 26 in the length direction (second direction) is not greater than 2 μm.

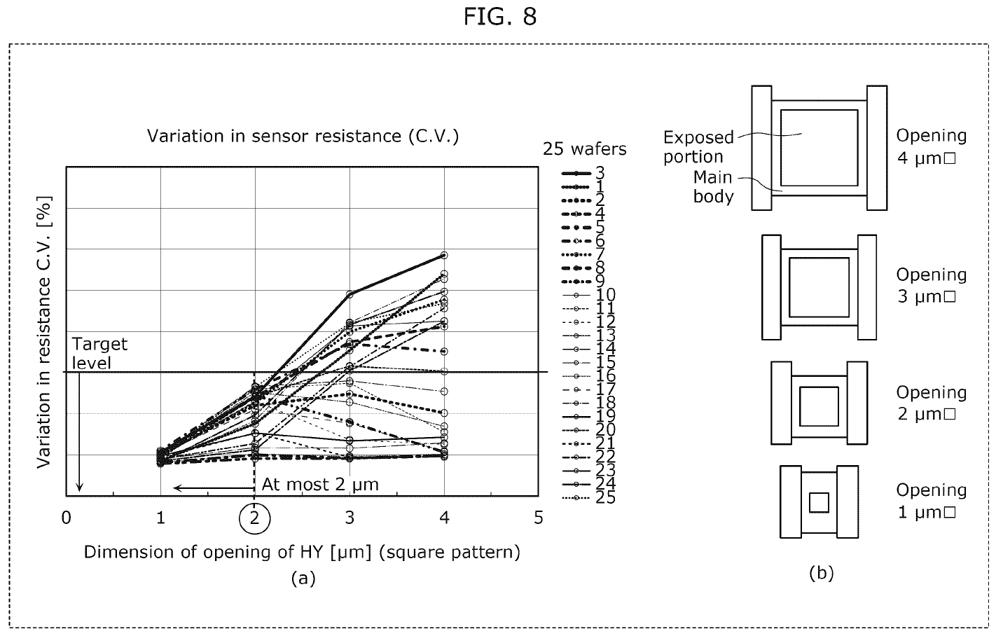

FIG. 8 is a diagram for describing the result of an experiment regarding dependency between dimension of opening of exposed portion and variation in resistance among hydrogen detection elements each of which includes exposed portion in square pattern. More specifically, (a) in FIG. 8 is a diagram illustrating the result of the experiment regarding dependency between dimension of opening of exposed portion and variation in sensor resistance measured for hydrogen detection elements each of which includes exposed portion in square pattern. The horizontal axis represents dimension of opening (length (μm) of one side of square pattern) of exposed portion, and the vertical axis represents variation (C.V. (%) in the same manner as FIG. 6) in resistance among 60 hydrogen detection elements that includes exposed portions and have been manufactured from a single wafer. In (a) in FIG. 8, data items regarding 60 hydrogen detection elements obtained from each of 25 wafers are plotted, as shown by the legend. (b) in FIG. 8 illustrates a top view (an exposed portion and a main body) of each of hydrogen detection elements that include exposed portions in four square pattern types (4 μm □, 3 μm □, 2 μm □, and 1 μm □). A dimension of a main body (the length of one side of a square) is a dimension of an exposed portion+1 μm.

As can be seen from FIG. 8, the smaller the size of a square pattern of an exposed portion is, the smaller variation in resistance among hydrogen detection elements is. Variation in resistance is desired to be not more than the “target level” illustrated in the figure, for suppressing variation in resistance to satisfy the specification realistically required for a hydrogen detection element. In other words, a dimension of a square pattern (i.e., the dimension of one side) of an exposed portion is preferably not greater than 2 μm.

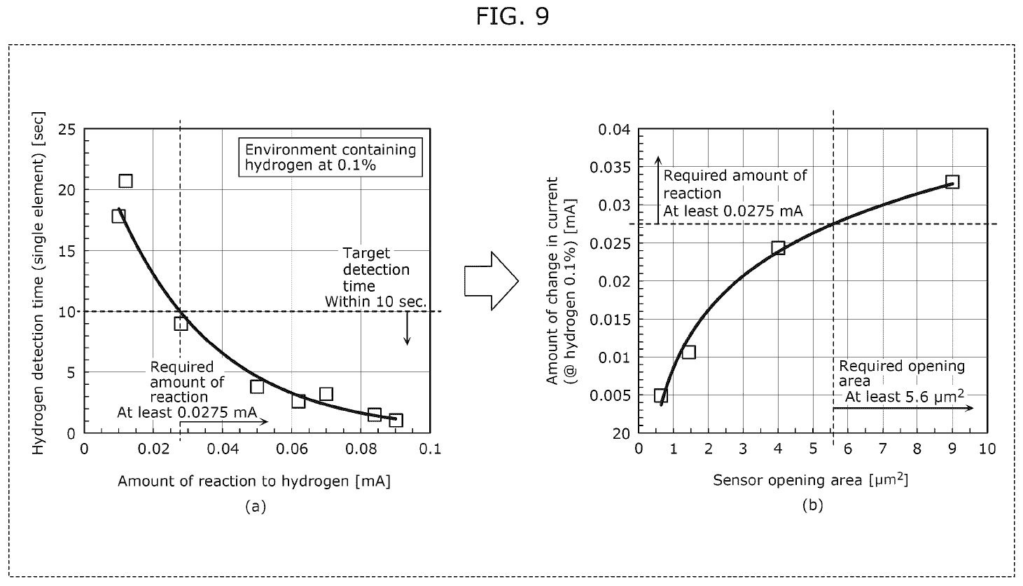

FIG. 9 is a diagram for describing the result of an experiment regarding a total opening area of exposed portions included in a single hydrogen detection element according to the embodiment. Here, FIG. 9 shows reaction characteristic when hydrogen at a concentration of 0.1% was introduced to hydrogen detection elements having various total opening areas of exposed portions. More specifically, (a) in FIG. 9 shows the relationship between detection time (the vertical axis: “hydrogen detection time (single element) (sec)”) and sensor current measured when hydrogen at a concentration of 0.1% was introduced to each of the hydrogen detection elements having various total opening areas of exposed portions (the horizontal axis: “amount of reaction to hydrogen (mA)”). (b) in FIG. 9 shows a graph in which data of the result of the experiment illustrated in (a) in FIG. 9 was rewritten as data showing the relationship between sensor current (the vertical axis: amount of change in current (@ hydrogen 0.1%) (mA)) and opening area of exposed portions included in hydrogen detection element (the horizontal axis: “sensor opening area (μm²)”).

It can be seen that an amount of reaction to hydrogen is required to be at least 0.0275 mA when the detection time is the “target detection time” that is within 10 seconds, considering the realistic specification, as illustrated in (a) in FIG. 9.

Moreover, it can be seen that a total opening area of exposed portions is required to be at least 5.6 μm² for ensuring an amount of reaction to hydrogen of at least 0.0275 mA, as illustrated in (b) in FIG. 9.

It can be seen from FIG. 9 that a total opening area of exposed portions 26 included in a hydrogen detection element according to the embodiment is preferably not less than 5.6 μm².

As described above, hydrogen detection element 10 according to the embodiment includes: first electrode 21 that is planar; second electrode 23 that is planar, is disposed opposite to first electrode 21, includes a principal surface covered by an insulating film (protective film 14 and second insulating film 13), and includes a plurality of exposed portions 26 that serve as a plurality of hydrogen gas inlets and are each provided by opening part of the insulating film on the principal surface; metal oxide layer 22 that is disposed between first electrode 21 and second electrode 23; and first terminal 25a and second terminal 25b that are electrically connected to second electrode 23 at positions between which the plurality of exposed portions 26 are arranged in plan view of second electrode 23. When hydrogen gas is introduced to the plurality of exposed portions 26, resistance between first terminal 25a and second terminal 25b changes.

Accordingly, by providing a plurality of exposed portions 26 to a single hydrogen detection element 10, a total opening area of the plurality of exposed portions 26 can be ensured to the same extent as that of the conventional hydrogen detection element while making the size of each exposed portion 26 smaller than that of conventional exposed portion 26. Thus, a hydrogen detection element having a characteristic structure for suppressing variation in reaction characteristic is realized.

Here, the plurality of exposed portions 26 are identical to each other in shape in the plan view of second electrode 23. For example, the shape may be a rectangular shape or an oval shape. Accordingly, a mask pattern for forming the plurality of exposed portions 26 is simplified.

Moreover, a maximum dimension in a second direction of each of the plurality of exposed portions 26 may be identical to a maximum dimension in a first direction of each of the plurality of exposed portions 26, the first direction connecting first terminal 25a and second terminal 25b, the second direction being perpendicular to the first direction and parallel to the principal surface. Accordingly, the plurality of exposed portions 26 each of which has a length and a width that are identical to each other are provided.

Moreover, the maximum dimension in the second direction of each of the plurality of exposed portions 26 may be smaller than the maximum dimension in the first direction of each of the plurality of exposed portions 26, the first direction connecting first terminal 25a and second terminal 25b, the second direction being perpendicular to the first direction and parallel to the principal surface. Accordingly, the plurality of exposed portions 26 that are elongated in the current direction are provided.

Moreover, the maximum dimension in the second direction of each of the plurality of exposed portions 26 may be at most 2 μm, the second direction being parallel to the principal surface and perpendicular to the first direction connecting first terminal 25a and second terminal 25b. Accordingly, variation in resistance can be suppressed to satisfy the specification realistically required for a hydrogen detection element.

Moreover, the plurality of exposed portions 26 may include a plurality of exposed portions 26 aligned in the first direction connecting first terminal 25a and second terminal 25b, or a plurality of exposed portions 26 aligned in the second direction being parallel to the principal surface and perpendicular to the first direction connecting first terminal 25a and second terminal 25b. Accordingly, a hydrogen detection element that includes a plurality of exposed portions 26 and has a characteristic structure for suppressing variation in reaction characteristic is realized.

Moreover, a method for manufacturing hydrogen detection element 10 according to the embodiment includes: forming first electrode 21 that is planar; forming metal oxide layer 22 on first electrode 21; forming second electrode 23 on metal oxide layer 22; forming first terminal 25a and second terminal 25b that are electrically connected to second electrode 23; forming an insulating film that covers second electrode 23; and forming, on a principal surface of second electrode 23, a plurality of exposed portions 26 that serve as a plurality of hydrogen gas inlets, by removing a plurality of portions of the insulating film between first terminal 25a and second terminal 25b in plan view of second electrode 23. When hydrogen gas is introduced to the plurality of exposed portions 26, resistance between first terminal 25a and second terminal 25b changes.

Accordingly, by providing a plurality of exposed portions 26 to a single hydrogen detection element 10, a total opening area of the plurality of exposed portions 26 can be ensured to the same extent as that of the conventional hydrogen detection element while making the size of each exposed portion 26 smaller than that of conventional exposed portion 26. Thus, a method for manufacturing a hydrogen detection element having a characteristic structure for suppressing variation in reaction characteristic is realized.

Hereinabove, although a hydrogen detection device according to the present disclosure and a manufacturing method thereof have been described based on the embodiment, the present disclosure is not limited to the embodiment. Various modifications of the embodiment as well as other embodiments resulting from combinations of some of the constituent elements from the embodiment that may be conceived by those skilled in the art are included within the scope of the present disclosure as long as they do not depart from the essence of the present disclosure.

For example, although an oval shape of each of the plurality of exposed portions 26 is elongated in the current direction in FIG. 2A to FIG. 2C, the oval shape is not limited to such an oval shape and may be an oval shape elongated in a direction orthogonal to the current direction.

Moreover, although the plurality of exposed portions 26 are identical to each other in shape and size in each hydrogen detection element in FIG. 2A to FIG. 2C, at least one of the shape or the size may be different among the plurality of exposed portions 26 in each hydrogen detection element.

Although an exemplary embodiment of the present disclosure has been described in detail above, those skilled in the art will readily appreciate that many modifications are possible in the exemplary embodiment without materially departing from the novel teachings and advantages of the present disclosure. Accordingly, all such modifications are intended to be included within the scope of the present disclosure.

### INDUSTRIAL APPLICABILITY

A hydrogen detection element according to the present disclosure can be used as a hydrogen detection element having a characteristic structure for suppressing variation in reaction characteristic, for example, as a hydrogen sensor used in a fuel-cell vehicle, a hydrogen station, a hydrogen plant, or the like.

1. A hydrogen detection element comprising:
a first electrode that is planar;
a second electrode that is planar, is disposed opposite to the first electrode, includes a principal surface covered by an insulating film, and includes a plurality of exposed portions that each serve as a hydrogen gas inlet and are each provided by opening part of the insulating film on the principal surface;
a metal oxide layer that is disposed between the first electrode and the second electrode; and
a first terminal and a second terminal that are electrically connected to the second electrode at positions between which the plurality of exposed portions are arranged in plan view of the second electrode, wherein
when hydrogen gas is introduced to the plurality of exposed portions, resistance between the first terminal and the second terminal changes.

2. The hydrogen detection element according to claim 1, wherein
the plurality of exposed portions are identical to each other in shape in the plan view of the second electrode.

3. The hydrogen detection element according to claim 2, wherein
the shape is a rectangular shape or an oval shape.

4. The hydrogen detection element according to claim 1, wherein
a maximum dimension in a second direction of each of the plurality of exposed portions is identical to a maximum dimension in a first direction of each of the plurality of exposed portions, the first direction connecting the first terminal and the second terminal, the second direction being perpendicular to the first direction and parallel to the principal surface.

5. The hydrogen detection element according to claim 1, wherein
a maximum dimension in a second direction of each of the plurality of exposed portions is smaller than a maximum dimension in a first direction of each of the plurality of exposed portions, the first direction connecting the first terminal and the second terminal, the second direction being perpendicular to the first direction and parallel to the principal surface.

6. The hydrogen detection element according to claim 1, wherein
a maximum dimension in a second direction of each of the plurality of exposed portions is at most 2 μm, the second direction being parallel to the principal surface and perpendicular to a first direction connecting the first terminal and the second terminal.

7. The hydrogen detection element according to claim 1, wherein
the plurality of exposed portions include a plurality of exposed portions aligned in a first direction connecting the first terminal and the second terminal.

8. The hydrogen detection element according to claim 1, wherein
the plurality of exposed portions include a plurality of exposed portions aligned in a second direction being parallel to the principal surface and perpendicular to a first direction connecting the first terminal and the second terminal.

9. A method for manufacturing a hydrogen detection element, the method comprising:
forming a first electrode that is planar;
forming a metal oxide layer on the first electrode;
forming a second electrode on the metal oxide layer;
forming a first terminal and a second terminal that are electrically connected to the second electrode;
forming an insulating film that covers the second electrode; and
forming, on a principal surface of the second electrode, a plurality of exposed portions that serve as a plurality of hydrogen gas inlets, by removing a plurality of portions of the insulating film between the first terminal and the second terminal in plan view of the second electrode, wherein
when hydrogen gas is introduced to the plurality of exposed portions, resistance between the first terminal and the second terminal changes.
